(19) United States
(12) Patent Application Publication
(10) Pub. No.: US 2008/0221423 A1
(43) Pub. Date: Sep. 11, 2008

(54) Cardiac Catheter Imaging System

(76) Inventor: Dirar S. Khoury, Houston, TX (US)

Correspondence Address: LAW OFFICES OF MARK L. BERRIER, 3811 BEE CAVES ROAD, SUITE 204, AUSTIN, TX, 78746, US

(21) Appl. No.: 11/681,860

(22) Filed: Mar. 5, 2007

(51) Int. Cl. A61B 5/04

(52) U.S. Cl. 600/374

(57) ABSTRACT

Systems and methods for measuring electrical potentials and other data associated with body tissue and generating electrograms of the tissue based on the data. In one embodiment, a device for measuring parameters of human tissue includes a multielectrode catheter for taking multiple measurements of the electrical characteristics of the human tissue, a concentric tube catheter located inside the multielectrode catheter, for providing structural support to the multi-electrode catheter and for serving as a conduit for advancing or withdrawing the multielectrode catheter over its surface; and an imaging catheter located inside the concentric tube catheter for taking multiple measurements of anatomical characteristics of the human tissue.

### CROSS-REFERENCE TO RELATED APPLICATIONS

This application claims the benefit of U.S. patent application Ser. No. 10/256,188, filed Sep. 26, 2002, which claims the benefit of U.S. Provisional Patent Application 60/325,707, filed Sep. 28, 2001, each of which is incorporated by reference as if set forth herein in its entirety.

### BACKGROUND

1. Field of the Invention

The invention relates generally to medical devices and methods, and more particularly to catheters that can be used to measure electrical potentials and other data associated with body tissue, wherein the data can then be used to generate electrograms of the tissue.

2. Related Art

Heart rhythm disorders (atrial and ventricular arrhythmias) result in significant morbidity and mortality. Unfortunately, current pharmacological therapy for managing cardiac arrhythmias is often ineffective and, at times, can cause arrhythmias, thereby shifting emphasis to nonpharmacological therapy (such as ablation, pacing, and defibrillation). Due to limitations in present mapping techniques, brief, chaotic, or complex arrhythmias (such as atrial fibrillation and ventricular tachycardia) cannot be mapped adequately during catheterization, resulting in unsuccessful elimination of the arrhythmia. In addition, localizing abnormal beats and delivering and quantifying the effects of therapy such as ablation are very time consuming during catheterization. Selecting appropriate pharmacological therapies and advancing nonpharmacological methods to manage cardiac arrhythmias are contingent on developing mapping techniques that identify mechanisms of arrhythmias, localize their sites of origin with respect to underlying cardiac anatomy, and elucidate effects of therapy. Therefore, to successfully manage cardiac arrhythmias, electrical-anatomical imaging on a beat-by-beat basis, simultaneously, and at multiple sites is required.

Electrical mapping of the heartbeat, whereby multielectrode arrays are placed on the exterior surface of the heart (epicardium) to directly record the electrical activity, has been applied extensively in both animals and humans. Although epicardial mapping provides detailed information on sites of origin and mechanisms of abnormal heart rhythms (arrhythmias), its clinical application has great limitation: it is performed at the expense of open-chest surgery. In addition, epicardial mapping does not provide access to interior heart structures that play critical roles in the initiation and maintenance of abnormal heartbeats.

Many heart rhythm abnormalities (arrhythmias) originate from interior heart tissues (endocardium). Further, because the endocardium is more safely accessible (without surgery) than the epicardium, most electrical mapping techniques and delivery of nonpharmacological therapies (e.g. pacing and catheter ablation) have focused on endocardial approaches by catheterization. However, current endocardial mapping techniques have certain limitations. Traditional electrode-catheter mapping performed during electrophysiology catheterization procedures is confined to a limited number of recording sites, is time consuming, and is carried out over several heartbeats without accounting for possible beat-to-beat variability in activation. While other catheter-mapping approaches provide important three-dimensional positions of a roving electrode-catheter through the use of “special” sensors, mapping is still performed over several heartbeats. On the other hand, although multielectrode basket-catheters measure endocardial electrical activities at multiple sites simultaneously by expanding the basket inside the heart so that the electrodes are in direct contact with the endocardium, the basket is limited to a fixed number of recording sites, may not be in contact with the entire endocardium, and may result in irritation of the myocardium.

An alternative mapping approach utilizes a noncontact, multielectrode cavitary probe that measures electrical activities (electrograms) from inside the blood-filled heart cavity from multiple directions simultaneously. The probe electrodes are not necessarily in direct contact with the endocardium; consequently, noncontact sensing results in a smoothed electrical potential pattern. Nonsurgical insertion of a noncontact, multielectrode balloon-catheter, that does not occlude the blood-filled cavity, has been reported in humans.

Present mapping systems cannot provide true images of endocardial anatomy during catheterization. Present systems often delineate anatomical features based on (1) extensive use of fluoroscopy; (2) deployment of multiple catheters, or roving the catheters, at multiple locations; and (3) assumptions about properties of recorded electrograms in relation to underlying anatomy (e.g. electrograms facing a valve are low in amplitude). However, direct correlation between endocardial activation and cardiac anatomy is important in order to clearly identify the anatomical sources of abnormal heartbeats, to understand the mechanisms of cardiac arrhythmias and their sequences of activation within or around complex anatomical structures, and to deliver appropriate therapy.

Early applications of the “inverse problem” of electrocardiography sought to noninvasively reconstruct (compute) epicardial surface potentials (electrograms) and activation sequences of the heartbeat based on noncontact potentials measured at multiple sites on the body surface. The computed epicardial potentials were in turn used to delineate information on cardiac sources within the underlying myocardium. To solve the “inverse problem”, numeric techniques have been repeatedly tested on computer, animal, and human models. Similarly, computing endocardial surface electrical potentials (electrograms) based on noncontact potentials (electrograms) measured with the use of a multielectrode cavitary probe constitutes a form of endocardial electrocardiographic “inverse problem.”

The objective of the endocardial electrocardiographic “inverse problem” is to compute virtual endocardial surface electrograms based on noncontact cavitary electrograms measured by multielectrode probes. Methods for acquisition of cavitary electrograms and computation of endocardial electrograms in the beating heart have been established and their accuracy globally confirmed. Determining the probe-endocardium geometrical relationship (i.e. probe position and orientation with respect to the endocardial surface) is required to solve the “inverse problem” and a prerequisite for accurate noncontact electrical-anatomical imaging. In previous studies, fluoroscopic imaging provided a means for beat-by-beat global validation of computed endocardial activation in the intact, beating heart. Furthermore, epicardial echocardiography was used to determine the probe-cavity geometrical model. However, complex geometry, such as that of the atrium, may not be easily characterized by transthoracic or epicardial echocardiography.

Accurate three-dimensional positioning of electrode-catheters at abnormal electrogram or ablation sites on the endocardium and repositioning of the catheters at specific sites are important for the success of ablation. The disadvantages of routine fluoroscopy during catheterization include radiation effects and limited three-dimensional localization of the catheter. New catheter-systems achieve better three-dimensional positioning by (1) using a specialized magnetic sensor at the tip of the catheter that determines its location with respect to an externally applied magnetic field, (2) calculating the distances between a roving intracardiac catheter and a reference catheter, each carrying multiple ultrasonic transducers, (3) measuring the field strength at the catheter tip-electrode, while applying three orthogonal currents through the patient's body to locate the catheter; and (4) emitting a low-current locator signal from the catheter tip and determining its distance from a multielectrode cavitary probe. With these mapping techniques true three-dimensional imaging of important endocardial anatomical structures is not readily integrated (only semi-realistic geometric approximations of the endocardial surface), and assumptions must often be made about properties of recorded electrograms in relation to underlying anatomy (e.g. electrograms facing the tricuspid and mitral annuli are low in amplitude).

### SUMMARY OF THE INVENTION

This disclosure is directed to systems and methods for use in measuring electrical potentials and other data associated with body tissue, and using the data to generate electrograms of the tissue, wherein one or more of the problems discussed above are solved.

For example, systems and methods are described that make possible the combined use of (1) a lumen-catheter carrying a plurality of sensing electrodes (multielectrode catheter-probe) for taking multiple noncontact and contact measurements, from different directions, of the electrical characteristics of interior tissue such as the heart (endocardium) and (2) an internal coaxial catheter carrying one or more imaging elements for visualizing the anatomical characteristics of the tissue. A middle coaxial lumen-catheter (sheath) provides structural support and serves as a conduit for advancing or withdrawing the multielectrode catheter over its surface, or inserting the anatomical imaging catheter through its lumen. The imaging catheter is inserted inside the multielectrode catheter-probe (or the supporting lumen-catheter when in use) and is moved to detect the tissue from inside the lumen using different modalities such as ultrasound, infrared, and magnetic resonance. Both the electrical and anatomical measurements are sent to a data acquisition system that in turn provides combined electrical and anatomical graphical or numerical displays to the operator.

Another feature of one embodiment is that the catheter imaging system simultaneously maps multiple interior heart surface electrical activities (endocardial electrograms) on a beat-by-beat basis and combines three-dimensional activation-recovery sequences with endocardial anatomy. Electrical-anatomical imaging of the heart, based on (1) cavitary electrograms that are measured with a noncontact, multielectrode probe and (2) three-dimensional endocardial anatomy that is determined with an integrated anatomical imaging modality (such as intracardiac echocardiography), provides an effective and efficient means to diagnose abnormal heartbeats and deliver therapy.

Another feature of one embodiment is that the integrated electrical-anatomical imaging catheter system contains both a multielectrode probe and an anatomical imaging catheter, which can be percutaneously introduced into the heart in ways similar to standard catheters used in routine procedures. This “noncontact” imaging approach reconstructs endocardial surface electrograms from measured probe electrograms, provides three-dimensional images of cardiac anatomy, and integrates the electrical and anatomical images to produce three-dimensional isopotential and isochronal images.

Another feature of one embodiment is that the method improves the understanding of the mechanisms of initiation, maintenance, and termination of abnormal heartbeats, which could lead to selecting or developing better pharmacological or nonpharmacological therapies. Mapping is conducted with little use of fluoroscopy on a beat-by-beat basis, and allows the study of brief, rare, or even chaotic rhythm disorders that are difficult to manage with existing techniques.

Another feature of one embodiment is that there is a means to navigate standard diagnostic-therapeutic catheters, and accurately guide them to regions of interest within an anatomically-realistic model of the heart that is derived from ultrasound, infrared, or magnetic resonance. The various embodiments of the present invention may provide considerable advantages in guiding clinical, interventional electrophysiology procedures, such as imaging anatomical structures, confirming electrode-tissue contact, monitoring ablation lesions, and providing hemodynamic assessment.

Another feature of one embodiment is that some of the sensing electrodes on the surface of the multielectrode catheter-probe are brought in direct contact with the interior surface of the tissue. The multielectrode catheter simultaneously measures contact and noncontact potentials resulting from electrical activity from multiple locations in the tissue.

Another feature of one embodiment is that the multielectrode catheter-probe is navigated inside a blood-filled cavity and placed at different locations. Meanwhile, the multielectrode catheter continuously measures contact and noncontact potentials resulting from electrical activity from multiple locations in the tissue.

Numerous other embodiments are also possible.

### BRIEF DESCRIPTION OF THE DRAWINGS

Other objects and advantages of the invention may become apparent upon reading the following detailed description and upon reference to the accompanying drawings.

FIG. 1 illustrates a system in accordance with one embodiment of the present invention in use with a human patient.

FIG. 2 illustrates a lumen sheath with a pig-tail at its distal end and a guide wire inside its lumen.

FIG. 3A illustrates a multielectrode catheter-probe with a lumen inside its shaft.

FIG. 3B illustrates an alternative embodiment of a multielectrode lumen catheter-probe whereby a grid of electrodes can be expanded.

FIG. 3C illustrates an alternative embodiment of a multielectrode lumen catheter-probe with a pig-tail at its distal end for structural support.

FIG. 4 illustrates an anatomical imaging catheter such as intracardiac echocardiography catheter.

FIG. 5A illustrates a configuration that combines the sheath (of FIG. 2) with the multielectrode catheter-probe (of FIG. 3A) over its surface at the proximal end and the anatomical imaging catheter (of FIG. 4) advanced inside the lumen at the distal end.

FIG. 5B illustrates an alternative embodiment that combines the sheath (of FIG. 2) with the multielectrode catheter-probe (of FIG. 3B) advanced over its surface to the distal end and the anatomical imaging catheter (of FIG. 4) inside the lumen at the proximal end.

FIG. 6 illustrates an alternative embodiment that combines the multielectrode catheter-probe (of FIG. 3C) with the anatomical imaging catheter (of FIG. 4) inside its lumen.

While the invention is subject to various modifications and alternative forms, specific embodiments thereof are shown by way of example in the drawings and the accompanying detailed description. It should be understood, however, that the drawings and detailed description are not intended to limit the invention to the particular embodiment which is described. This disclosure is instead intended to cover all modifications, equivalents and alternatives falling within the scope of the present invention as defined by the appended claims.

### DETAILED DESCRIPTION OF PREFERRED EMBODIMENTS

One or more embodiments of the invention are described below. It should be noted that these and any other embodiments described below are exemplary and are intended to be illustrative of the invention rather than limiting.

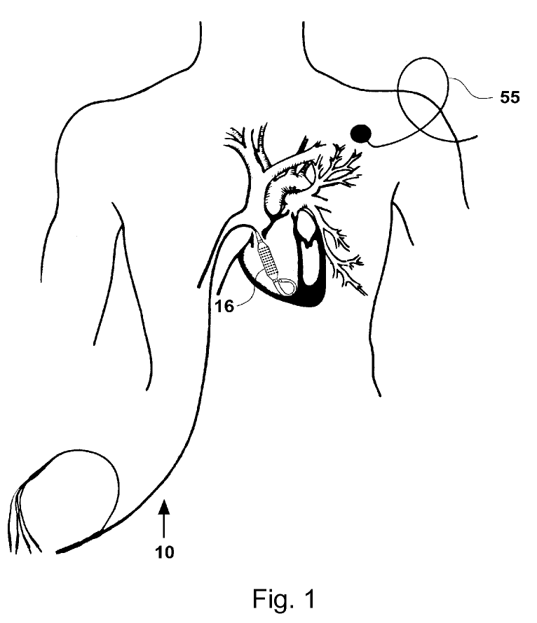

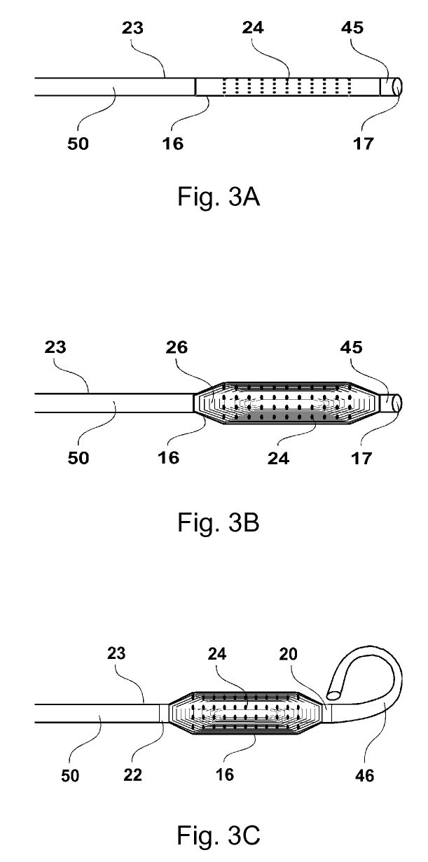

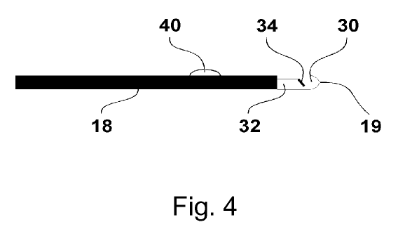

FIG. 1 illustrates an electrical-anatomical imaging catheter-system 10 in use in a human patient. The catheter is percutaneously inserted through a blood vessel (vein or artery) and advanced into the heart cavity. The catheter detects both electrical and anatomical properties of interior heart tissue (endocardium). Measured electrical properties are in the form of contact and noncontact potentials detected by electrodes (sensors) 24 (illustrated in FIG. 3A). Measured anatomical properties are in the form of tissue geometry, structure, and texture features detected by an anatomical imaging catheter 18 (illustrated in FIG. 4).

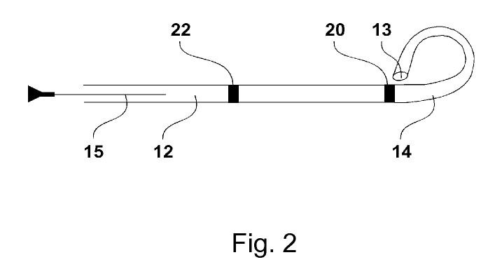

Referring now to FIG. 2, the electrical-anatomical imaging catheter system 10 includes a lumen sheath 12 (about 3 mm in diameter) which has a pig tail distal end 14 to minimize motion artifacts inside the heart cavity. A guide wire 15 is advanced to a tip 13 to guide the sheath 12. The sheath 12 provides structural support for a coaxial multielectrode catheter-probe 16 (illustrated in FIG. 3A and FIG. 3B) that slides over the surface of the sheath 12, and records noncontact cavitary electrical signals (electrograms) from multiple directions and at several locations along the sheath. The sheath 12 also functions as a conduit for inserting an anatomical imaging catheter 18 (illustrated in FIG. 4) such as a standard intracardiac echocardiography (ICE) catheter that records continuous echocardiographic images of the heart interior. With this approach, the sheath 12 maintains the same imaging axis and direction over several deployments inside the heart cavity of both the probe 16 and the anatomical imaging catheter 18. Radiopaque and sonopaque ring marker 20 at the distal end of the sheath 12 and radiopaque and sonopaque ring marker 22 at the proximal end of the sheath 12 aid in verifying the probe 16 and the anatomical imaging catheter 18 locations.

Referring now to FIG. 3A, the electrical-anatomical imaging catheter system 10 includes a lumen catheter which carries a plurality of sensing electrodes 24 on its surface that make up the multielectrode probe 16. The electrodes 24 are arranged in columns. The diameter of the probe 16 is similar to that of shaft 23 of the probe 16(on the order of 3 mm). The sheath 12 and the anatomical imaging catheter 18 both coaxially fit inside the lumen of the probe 16. The catheter-probe 16 has a straight distal end 45 that permits sliding the probe 16 over the coaxial lumen sheath 12. In this state the probe 16 is easily inserted percutaneously by the operator through a blood vessel and advanced into the heart cavity. By sliding the catheter-probe 16 over the central sheath 12, it is possible to place the probe 16 at multiple locations over the sheath and along the axis of the cavity. The shaft 23 of the probe 16 is shorter than the central sheath 12 so that it slides easily over the sheath 12 in and out of the heart cavity.

FIG. 3B illustrates another embodiment of part of the electrical anatomical imaging catheter-system 10 of the present system, in which for the probe 16, the electrodes 24 are laid on a central balloon 26 that is inflated to a fixed diameter without the electrodes 24 necessarily touching the interior surface of the heart. The balloon is similar to angioplasty catheters used in routine catheterization procedures. The balloon 26 is inflated inside the heart cavity to enlarge the probe 16. The sheath 12 and the anatomical imaging catheter 18 (illustrated in FIG. 4) fit inside the lumen 50. The probe 16 has a straight distal end 45 that permits sliding the probe 16 over the coaxial lumen sheath 12. By sliding the probe 16 over the central sheath 12, it is possible to place the probe 16 at multiple locations over the sheath and along the axis of the heart cavity. In its collapsed state the size of the probe 16 is similar to that of the sheath 12. Thus, the operator is able to insert the probe percutaneously and inflate it inside the heart without occluding the cavity. The shaft 23 of the probe 16 is shorter than the central sheath 12 so that the probe 16 slides easily over the sheath 12 in and out of the cavity.

In another embodiment of the electrical-anatomical imaging catheter system 10, FIG. 3C illustrates the probe 16 with a pig-tail 46 at its distal end to minimize motion artifacts of the probe 16. In this embodiment, the probe 16 is used independently of the lumen sheath 12. The anatomical imaging catheter 18 (illustrated in FIG. 4) fits inside the lumen of the probe 16.

Referring now to FIG. 4, the anatomical imaging catheter 18 is used to image interior structures of the heart. In the preferred embodiment, the catheter 1 8 is a 9-MHz intracardiac echocardiography catheter (Model Ultra ICE, manufactured by Boston Scientific/EPT, located in San Jose, Calif.). To acquire echocardiographic images, the catheter 18 connects to an imaging console (Model ClearView, manufactured by Boston Scientific/EPT, located in San Jose, Calif.). The catheter 18 has a distal imaging window 30 and a rotatable imaging core 32 with a distal transducer 34 that emits and receives ultrasound energy. Continuous rotation of the transducer provides tomographic sections of the heart cavity. The design of the present system allows for integrating other anatomical imaging catheters presently under development such as echocardiography catheters carrying multiple phased-array transducers, infrared, and magnetic resonance imaging catheters. While the anatomical imaging catheter 18 is in use, the three-dimensional anatomical reconstruction assumes that the catheter 18 is straight and thus straightens the image of the heart cavity. If the catheter 18 curves, the image is distorted, or, if the catheter 18 rotates during pullback, the image is twisted. Therefore, in the preferred embodiment, a position and orientation sensor 40 is added to the catheter 18.

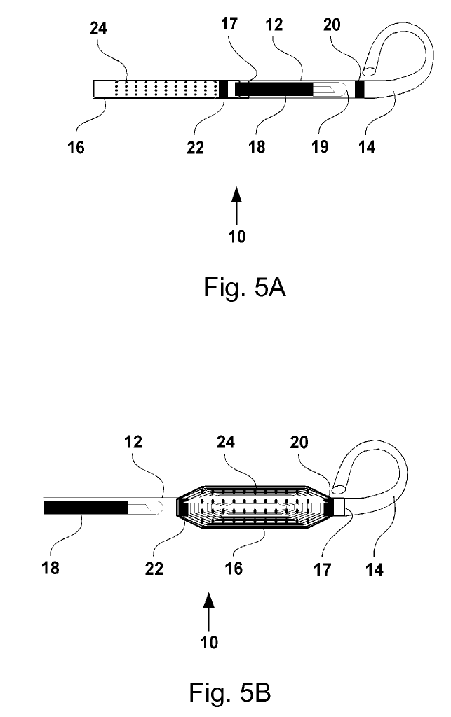

Referring now to FIG. 5A, an integrated, noncontact, electrical anatomical imaging catheter-system 10 is illustrated that combines the sheath 12 with the multielectrode catheter-probe 16 over its surface at the proximal end, and the anatomical imaging catheter 18 inside the lumen at the distal end. In operation, the probe 16 is preloaded over the central sheath 12, thereby enabling the probe 16 to move in and out of the heart cavity in small increments at several locations over a fixed axis. The guide wire 15 is placed inside the central sheath 12 to ensure the pig-tail end 14 remains straight during insertion through a blood vessel. With the probe 16 loaded on the sheath 12 and pulled back, the sheath 12 is advanced through a blood vessel and placed inside the heart cavity under the guidance of fluoroscopy, and the guide wire 15 is then removed. The anatomical imaging catheter 18 is then inserted through the lumen of the central sheath 12, replacing the guide wire 15, and advanced until a tip 19 of the catheter 18 is situated at the pre-determined radiopaque and sonopaque distal marker 20 on the sheath 12. The catheter 18 is pulled back from the distal marker 20 to the proximal marker 22 on the sheath 12 at fixed intervals, and noncontact anatomical images are continuously acquired at each interval.

Referring now to FIG. 5B, under the guidance of fluoroscopy, the probe 16 is advanced over the central sheath 12 until a tip 17 is at the distal marker 20, and the balloon 26 (if used) is inflated to unfold the probe 16. The probe 16 then simultaneously acquires noncontact cavitary electrograms.

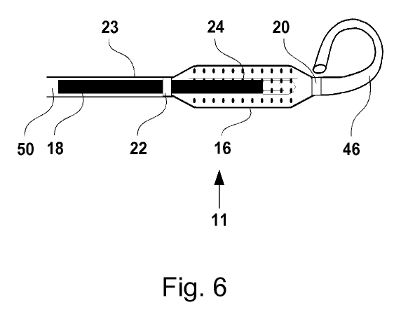

Referring now to FIG. 6, an alternate embodiment of the integrated electrical-anatomical imaging catheter system 10 is illustrated, labeled as an integrated electrical-anatomical imaging catheter system 11, in which the lumen sheath 12 is eliminated. A multielectrode lumen catheter-probe 16 with a pig-tail 46 at its distal end is inserted inside the heart cavity and is used to acquire noncontact electrograms. In operation, the multielectrode catheter-probe is navigated inside the cavity and placed at different locations. The anatomical imaging catheter 18 is inserted inside the lumen of the catheter-probe 16, and imaging is performed from inside the probe 16.

Unipolar cavitary electrograms sensed by the noncontact multielectrode probe 16 with respect to an external reference electrode 55 (shown in FIG. 1) along with body surface electrocardiogram signals, are simultaneously acquired with a computer-based multichannel data acquisition mapping system, which, in the preferred embodiment, is the one built by Prucka Engineering-GE Medical Systems, located in Milwaukee, Wis. In operation, the multielectrode catheter-probe 16 senses both noncontact potentials (electrograms) by electrodes 24 not in contact with the tissue interior, and contact potentials (electrograms) by electrodes 24 in direct contact with the tissue interior. The mapping system amplifies and displays the signals at a 1 ms sampling interval per channel. The mapping system displays graphical isopotential and isochronal maps that enable evaluation of the quality of the data acquired during the procedure and interaction with the study conditions. The multiple anatomical images (such as ICE) are digitized, and the interior heart borders automatically delineated. The cavity three-dimensional geometry is rendered in a virtual reality environment, as this advances diagnostic and therapeutic procedures.

To reconstruct the electrical activities (electrical potentials, V) on the interior heart surface (endocardium) based on noncontact electrical potentials measured by the cavitary multielectrode probe 16 and anatomical information derived from the anatomical imaging catheter 18, Laplace's equation (F 2V=0) is numerically solved in the blood-filled cavity between the probe 16 and the endocardium. The boundary element method is employed in computing the electrical potentials at the tissue surface in a three-dimensional geometry on the basis of noncontact cavitary potentials sensed by electrodes 24. A numeric regularization technique (filtering) based on the commonly used Tikhonov method is employed to find the electrical potentials on the endocardium. Here, with the probe 16 positioned at one location inside the cavity, the electrical potentials are then uniquely reconstructed on the real endocardial anatomy derived from the anatomical imaging catheter 18.

Due to the irregular shape of the tissue and its continuous dynamic motion throughout the cardiac cycle, some of electrodes 24 may be in contact with the tissue. At other times, some of electrodes 24 may be intentionally placed in contact with the tissue when positioning the multielectrode probe 16 in complex regions of the cavity. Select electrodes 24 on the surface of the probe 16 that are in contact with the tissue, as identified by the anatomical imaging catheter 18, record contact electrical potentials. Meanwhile, the remainder of electrodes 24 on the surface of probe 16 measure noncontact potentials. Values of tissue contact potentials may be used as boundary conditions when numerically solving Laplace's equation (i.e. V=Vcontact at the interior tissue boundary). By applying the boundary element method and numeric regularization, the resulting solution is a set of electrical potentials at multiple locations throughout the tissue surface.

In cases of complex cavity geometry, the multielectrode probe 16 may be navigated to different locations inside the cavity. Meanwhile, electrodes 24 may record noncontact electrical potentials at multiple locations of probe 16, thereby providing a large number of spatial samples pf noncontact cavitary potentials that improve the accuracy of potentials computed at the interior tissue surface. The noncontact potentials recorded at multiple locations of probe 16 may be combined into one large set of data to simultaneously reconstruct the potentials at the tissue surface. Alternatively, potentials at the tissue surface may be repeatedly reconstructed on the basis of each individual location of probe 16 inside the cavity, with final tissue potentials computed as the average for all probe locations. In either approach, the potentials at the tissue surface continue to be reconstructed by numerically solving Laplace's equation and applying the boundary element method and numeric regularization.

Nonfluoroscopic three-dimensional positioning and visualization of standard navigational electrode-catheters is clinically necessary for (1) detailed and localized point-by-point mapping at select interior heart regions, (2) delivering nonpharmacological therapy such as pacing or ablation, (3) repositioning the catheters at specific sites, and (4) reducing the radiation effects of fluoroscopy during catheterization. To guide three-dimensional positioning and navigation of standard electrode-catheters, a low-amplitude location electrical signal is emitted between the catheter tip-electrode and the external reference electrode 55, and sensed by multiple electrodes 24 on the surface of the probe 16. The catheter tip is localized by finding the x, y, and z coordinates of a location point p. The location of the emitting electrode is determined by minimizing [F(p)−V(p)]T[F(p)−V(p)] with respect to p, where V(p) are the electrical potentials measured on the probe 16, and F(p) are the electrical potentials computed on the probe 16 using an analytical (known) function and assuming an infinite, homogeneous conducting medium. This process also constructs the shape of the catheter within the cavity by determining the locations of all catheter electrodes. Alternatively, the location and shape of the roving electrode-catheter is determined with respect to the underlying real anatomy by direct visualization with the anatomical imaging catheter 18.

The present method senses the location signal by multiple probe electrodes 24 simultaneously, thereby localizing the roving catheter more accurately than prior art methods. Furthermore, the method reconstructs the shape of the roving catheter during navigation by emitting a location signal from each of the catheter electrodes and determining their locations within the cavity. With this approach, online navigation of standard electrode-catheters is performed and displayed within an anatomically-correct geometry derived from ultrasound, infrared, or magnetic resonance, and without extensive use of fluoroscopy.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed within the following claims.

1-9. (canceled)

10. A device for measuring electrical and geometrical characteristics of body tissue from a blood-filled cavity within the tissue, comprising:
a multielectrode lumen catheter, having multiple electrodes arranged in a fixed pattern on a continuous surface, wherein the electrodes are configured to take multiple simultaneous contact and non-contact measurements of electrical potentials resulting from electrical activity from multiple locations in the tissue; and
an anatomical imaging catheter having at least one imaging element for visualizing anatomical characteristics located inside the multielectrode lumen catheter, wherein the anatomical imaging catheter is configured to take multiple non-contact measurements of anatomical characteristics of the tissue, and for determining location and orientation of the multielectrode lumen catheter with respect to the tissue;
wherein the multielectrode lumen catheter and the anatomical imaging catheter are configured to provide the measurements of electrical potentials and the measurements of anatomical characteristics to a data processing system for reconstruction of tissue electrograms.

11. The device of claim 1, further comprising a coaxial tube catheter located inside the multielectrode lumen catheter, wherein the coaxial tube catheter provides structural support to the multielectrode lumen catheter, and serves as a conduit for advancing or withdrawing the multielectrode lumen catheter over the surface of the coaxial tube catheter, and for advancing or withdrawing the anatomical imaging catheter within a lumen of the coaxial tube catheter.

12. The device of claim 10, wherein the multielectrode lumen catheter is configured to provide the contact and non-contact measurements of electrical potentials of the tissue while the multielectrode lumen catheter is navigated inside the blood-filled cavity and placed at different locations.

13. The device of claim 12, wherein the data processing system comprises a data acquisition system, a data analysis system, and a data display system coupled to the device,
wherein the data acquisition system is responsive to the multiple contact and non-contact measurements of the electrical potentials and the measurements of anatomical characteristics to provide electrical and anatomical data to the data analysis system,
wherein the data analysis system is responsive to the electrical and anatomical data to reconstruct the tissue surface electrograms by solving Laplace's equation, wherein Laplace's equation is solved by employing the boundary element method and numeric regularization, and
wherein the data display system is responsive to the tissue surface electrograms and anatomical data to depict three-dimensional electrical, anatomical, and functional characteristics of the tissue.

14. The device of claim 11, wherein the multielectrode lumen catheter is configured to provide the contact and non-contact measurements of electrical potentials of the tissue while the multielectrode lumen catheter is navigated inside the blood-filled cavity and placed at different locations.

15. The device of claim 14, wherein the data processing system comprises a data acquisition system, a data analysis system, and a data display system coupled to the device,
wherein the data acquisition system is responsive to the multiple contact and non-contact measurements of the electrical potentials and the measurements of anatomical characteristics to provide electrical and anatomical data to the data analysis system,
wherein the data analysis system is responsive to the electrical and anatomical data to reconstruct the tissue surface electrograms by solving Laplace's equation, wherein Laplace's equation is solved by employing the boundary element method and numeric regularization, and
wherein the data display system is responsive to the tissue surface electrograms and anatomical data to depict three-dimensional electrical, anatomical, and functional characteristics of the tissue.

16. A method for measuring electrical and geometrical characteristics of body tissue from a blood-filled cavity within the tissue, comprising:
inserting into the cavity a multielectrode lumen catheter having multiple electrodes arranged in a fixed pattern on a continuous surface;
inserting through the multielectrode lumen catheter and into the cavity an anatomical imaging catheter having at least one imaging element for visualizing anatomical characteristics;
determining location and orientation of the multielectrode lumen catheter with respect to the tissue using the imaging catheter;
taking multiple simultaneous contact and non-contact measurements of electrical potentials resulting from electrical activity from multiple locations in the tissue using the multielectrode lumen catheter;
taking multiple non-contact measurements of anatomical characteristics of the tissue using the imaging catheter; and
reconstructing tissue surface electrograms based on the determined location and orientation of the multielectrode lumen catheter with respect to the tissue, the measured electrical potentials and the measured anatomical characteristics.

17. The method of claim 5, wherein inserting the multielectrode lumen catheter and the anatomical imaging catheter into the cavity comprise:
sliding a coaxial tube catheter into the cavity;
sliding the multielectrode lumen catheter over the outside surface of the coaxial tube catheter and into the cavity; and
sliding the anatomical imaging catheter through the interior of the coaxial tube catheter and into the cavity.

18. The method of claim 16, further comprising navigating the multielectrode lumen catheter inside the blood-filled cavity and placing it at different locations while taking the multiple contact and non-contact measurements of electrical potentials resulting from electrical activity from multiple locations in the tissue.

19. The method of claim 18, wherein reconstructing the tissue surface electrograms comprises sending the multiple contact and non-contact measurements of the electrical potentials and the measurements of anatomical characteristics to a data processing system and reconstructing the tissue surface electrograms in the data processing system.

20. The method of claim 19, wherein reconstructing the tissue surface electrograms comprises numerically reconstructing three-dimensional electrical characteristics of the tissue by solving Laplace's equation based on the measurements of the electrical potentials and anatomical characteristics, and employing the boundary element method and numeric regularization.

21. The method of claim 17, further comprising navigating the multielectrode lumen catheter inside the blood-filled cavity and placing it at different locations while taking the multiple contact and non-contact measurements of electrical potentials resulting from electrical activity from multiple locations in the tissue.

22. The method of claim 21, wherein reconstructing the tissue surface electrograms comprises sending the multiple contact and non-contact measurements of the electrical potentials and the measurements of anatomical characteristics to a data processing system and reconstructing the tissue surface electrograms in the data processing system.

23. The method of claim 22, wherein reconstructing the tissue surface electrograms comprises numerically reconstructing three-dimensional electrical characteristics of the tissue by solving Laplace's equation based on the measurements of the electrical potentials and anatomical characteristics, and employing the boundary element method and numeric regularization.
